(19) United States
(12) Patent Application Publication
(10) Pub. No.: US 2026/0085027 A1
(43) Pub. Date: Mar. 26, 2026

(54) METHOD OF PRODUCING HYDROFLUOROOLEFIN

(71) Applicant: AGC INC., Tokyo (JP)

(72) Inventors: Hikaru IWASAKI, Tokyo (JP); Taku YAMADA, Tokyo (JP); Tetsuo OTSUKA, Tokyo (JP)

(73) Assignee: AGC INC., Tokyo (JP)

(21) Appl. No.: 19/398,628

(22) Filed: Nov. 24, 2025

Related U.S. Application Data
(63) Continuation of application No. PCT/JP2024/013649, filed on Apr. 2, 2024.

(30) Foreign Application Priority Data
May 30, 2023 (JP) 2023-089279

(51) Int. Cl. C07C 17/25

(52) U.S. Cl. CPC C07C 17/25

(57) ABSTRACT

This method of producing a hydrofluoroolefin includes contacting a mixture including a hydrofluorocarbon and at least one selected from the group consisting of CX^{A1}X^{A2}═CX^{A3}X^{A4}, CX^{A5}≡CX^{A6}, and CX^{A7}X^{A8}X^{A9}—CX^{A10}X^{A11}X^{A12} with a catalyst, and obtaining the hydrofluoroolefin by a dehydrofluorination reaction of the hydrofluorocarbon, each of X^{A1} to X^{A12} independently represents a hydrogen atom, a fluorine atom, a chlorine atom, or a hydrocarbon chain having 1 to 8 carbon atoms, at least one of X^{A1} to X^{A4} has at least one of a hydrogen atom or a chlorine atom, at least one of X^{A5} or X^{A6} has at least one of a hydrogen atom or a chlorine atom, and at least one of X^{A7} to X^{A12} has a halogen atom other than a fluorine atom.

### CROSS-REFERENCE TO RELATED APPLICATIONS

This application is a Continuation Application of International Application No. PCT/JP2024/013649, filed Apr. 2, 2024, the disclosure of which is incorporated herein by reference in its entirety. Further, this application claims priority from Japanese Patent Application No. 2023-089279, filed May 30, 2023, the disclosure of which is incorporated herein by reference in its entirety.

### TECHNICAL FIELD

The present disclosure relates to a method of producing a hydrofluoroolefin.

### BACKGROUND ART

In recent years, hydrofluoroolefins have attracted attention as compounds having a low global warming potential.

As a method of producing a hydrofluoroolefin, for example, a method is known in which a hydrofluorocarbon is contacted with a catalyst such as a metal oxide to induce a dehydrofluorination reaction, thereby obtaining a hydrofluoroolefin (for example, Patent Literature 1).

### CITATION LIST

### Patent Literature

Patent Literature 1: Japanese Patent No. 6432526

### SUMMARY OF INVENTION

### Technical Problem

However, in a case in which a hydrofluorocarbon is contacted with a metal catalyst to undergo dehydrofluorination, there has been a tendency for the conversion rate to decrease during long-term production.

An object of one embodiment of the present invention is to provide a method of producing a hydrofluoroolefin in which a decrease in the conversion rate of the hydrofluorocarbon during long-term production is suppressed.

### Solution to Problem

The present disclosure includes the following aspects.
<1>

A method of producing a hydrofluoroolefin, the method including:
contacting a mixture including a hydrofluorocarbon and a compound A, which is at least one selected from the group consisting of a compound represented by the following Formula (A1), a compound represented by the following Formula (A2), and a compound represented by the following Formula (A3), with a catalyst, and
obtaining the hydrofluoroolefin by a dehydrofluorination reaction of the hydrofluorocarbon.

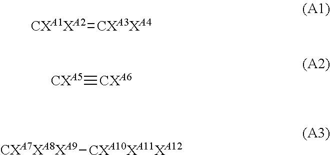

In Formulas (A1) and (A2), each of X^{A1}, X^{A2}, X^{A3}, X^{A4}, X^{A5}, and X^{A6} independently represents a hydrogen atom, a fluorine atom, a chlorine atom, or a hydrocarbon chain having 1 to 8 carbon atoms which may be substituted with at least one of a fluorine atom or a chlorine atom, at least one of X^{A1}, X^{A2}, X^{A3}, or X^{A4} has at least one of a hydrogen atom or a chlorine atom, and at least one of X^{A7} or X^{A6} has at least one of a hydrogen atom or a chlorine atom.

In Formula (A3), each of X^{A7}, X^{A8}, X^{A9}, X^{A10}, X^{A11}, and X^{A12} independently represents a hydrogen atom, a halogen atom, or a hydrocarbon chain having 1 to 8 carbon atoms which may be substituted with a halogen atom, and at least one of X⁷, X^{A8}, X^{A9}, X^{A10}, X^{A11}, or X^{A12} has a halogen atom other than a fluorine atom.
<2>

The method of producing a hydrofluoroolefin according to <1>, in which the compound A includes at least one selected from the group consisting of tetrachloroethane, trichloroethylene, trichloroethane, 1,2-dichloroethylene, and 1,1-dichloroethylene.
<3>

The method of producing a hydrofluoroolefin according to <1> or <2>, in which the hydrofluorocarbon is a compound represented by the following Formula (1), and the hydrofluoroolefin is a compound represented by the following Formula (2).

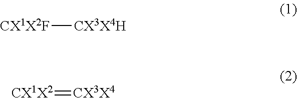

In Formulas (1) and (2), each of X¹, X², X³, and X⁴ independently represents a hydrogen atom or a fluorine atom, at least one of X¹, X², X³, or X⁴ is a fluorine atom, and at least one of X¹, X², X³, or X⁴ is a hydrogen atom.
<4>

The method of producing a hydrofluoroolefin according to <3>, in which the hydrofluorocarbon is at least one selected from the group consisting of 1,1,1-trifluoroethane, 1,1,2-trifluoroethane, 1,1,2,2-tetrafluoroethane, and 1,1,1,2-tetrafluoroethane, and the hydrofluoroolefin is at least one selected from the group consisting of 1,2-difluoroethylene, 1,1-difluoroethylene, and trifluoroethylene.
<5>

The method of producing a hydrofluoroolefin according to <3> or <4>, in which the compound A contains a compound represented by the following Formula (B1).

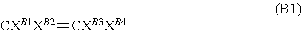

In Formula (B1), each of X^{B1}, X^{B2}, X^{B3}, and X^{B4} independently represents a group corresponding to X¹, X², X³, and X⁴ in Formulas (1) and (2), in a case in which each of X¹, X², X³, and X⁴ in Formulas (1) and (2) independently represents a hydrogen atom, X^{B1}, X^{B2}, X^{B3}, and X^{B4} are hydrogen atoms, and in a case in which each of X¹, X², X³, and X⁴ in Formulas (1) and (2) independently represents a fluorine atom, X^{B1}, X^{B2}, X^{B3}, and X^{B4} are chlorine atoms.
<6>

The method of producing a hydrofluoroolefin according to any one of <1> to <5>, in which the hydrofluorocarbon is at least one selected from the group consisting of 1,1,2,2-tetrafluoroethane and 1,1,1,2-tetrafluoroethane, and the compound A is trichloroethylene.
<7>

The method of producing a hydrofluoroolefin according to any one of <1> to <6>, in which a molar ratio between the compound A and the hydrofluorocarbon contained in the mixture is from 0.05 to 0.99.
<8>

The method of producing a hydrofluoroolefin according to any one of <1> to <7>, in which the mixture and the catalyst are contacted with each other at a temperature from 100 to 800° C.
<9>

The method of producing a hydrofluoroolefin according to any one of <1> to <8>, in which a contact time between the mixture and the catalyst is from 0.1 to 100.0 seconds.
<10>

The method of producing a hydrofluoroolefin according to any one of <1> to <9>, further including: drying the catalyst before contacting the mixture with the catalyst.
<11>

The method of producing a hydrofluoroolefin according to any one of <1> to <10>, in which a concentration of water in the mixture is less than 500 ppm.
<12>

The method of producing a hydrofluoroolefin according to any one of <1> to <11>, further including:
generating the hydrofluoroolefin and hydrogen fluoride by a dehydrofluorination reaction of the hydrofluorocarbon; and
causing the compound A to act on the hydrogen fluoride generated by the dehydrofluorination reaction of the hydrofluorocarbon.
<13>

The method of producing a hydrofluoroolefin according to <12>, in which, causing the compound A to act on the hydrogen fluoride generated by the dehydrofluorination reaction of the hydrofluorocarbon includes, in a case in which the compound A is a compound represented by the Formula (A1) or a compound represented by the Formula (A2), a hydrogen fluoride addition reaction occurring in which hydrogen fluoride is added to the compound A, and in a case in which the compound A is a compound represented by the Formula (A3), a halogen exchange reaction occurring in which a halogen atom other than a fluorine atom in the compound A is exchanged with a fluorine atom of the hydrogen fluoride.

### Advantageous Effects of Invention

According to the present disclosure, there is provided a method of producing a fluoroolefin in which a decrease in the conversion rate of the hydrofluorocarbon during long-term production is suppressed.

### DESCRIPTION OF EMBODIMENTS

In the disclosure, a numerical range indicated using “to” means a range including numerical values stated before and after “to” as a minimum value and a maximum value, respectively.

In a numerical range described in stages in the disclosure, an upper limit value or a lower limit value described in a certain numerical range may be replaced with an upper limit value or a lower limit value of another described numerical range in stages. In the numerical ranges described in the disclosure, an upper limit value or a lower limit value described in a certain numerical range may be replaced with a value shown in Examples.

In the disclosure, a combination of two or more preferable embodiments is a more preferable embodiment.

In the disclosure, when there are plural kinds of substances corresponding to each component, the amount of each component means the total amount of the plural kinds of substances unless otherwise specified.

### [Method of Producing a Hydrofluoroolefin]

A method of producing a hydrofluoroolefin of the disclosure includes: contacting a mixture including a hydrofluorocarbon and a compound A, which is at least one selected from the group consisting of a compound represented by the following Formula (A1), a compound represented by the following Formula (A2), and a compound represented by the following Formula (A3), with a catalyst, and obtaining the hydrofluoroolefin by a dehydrofluorination reaction of the hydrofluorocarbon.

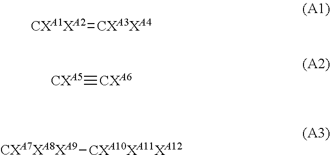

In Formulas (A1) and (A2), each of X^{A1}, X^{A2}, X^{A3}, X^{A4}, X^{A5}, and X^{A6} independently represents a hydrogen atom, a fluorine atom, a chlorine atom, or a hydrocarbon chain having 1 to 8 carbon atoms which may be substituted with at least one of a fluorine atom or a chlorine atom, at least one of X^{A1}, X^{A2}, X^{A3}, or X^{A4} has at least one of a hydrogen atom or a chlorine atom, and at least one of X^{A5} or X^{A6} has at least one of a hydrogen atom or a chlorine atom.

In Formula (A3), each of X^{A7}, X^{A8}, X^{A9}, X^{A10}, X^{A11}, and X^{A12} independently represents a hydrogen atom, a halogen atom, or a hydrocarbon chain having 1 to 8 carbon atoms which may be substituted with a halogen atom, and at least one of X^{A7}, X^{A8}, X^{A9}, X^{A10}, X^{A11}, or X^{A12} has a halogen atom other than a fluorine atom.

Hereinafter, hydrofluorocarbon is referred to as “HFC”, hydrofluoroolefin is referred to as “HFO”, a compound represented by Formula (A1) is referred to as “compound (A1)”, a compound represented by Formula (A2) is referred to as “compound (A2)”, a compound represented by Formula (A3) is referred to as “compound (A3)”, and at least one selected from the group consisting of compound (A1), compound (A2), and compound (A3) is also referred to as “compound A”.

According to the method of producing HFO of the disclosure, a decrease in the conversion rate of HFC during long-term production is suppressed. Although the reason for this is not clear, it is presumed as follows.

In the reaction for obtaining HFO by the dehydrofluorination reaction of HFC, hydrogen fluoride is generated. It is considered that the generated hydrogen fluoride reduces the catalytic activity. Specifically, the catalyst is fluorinated by hydrogen fluoride, the composition of the catalyst changes, and the crystal structure of the catalyst changes. A coking phenomenon occurs in which carbon-containing components accumulate on the surface of the catalyst due to factors such as the decomposition of the generated HFO or the like by hydrogen fluoride, leading to carbon deposition on the catalyst surface; and the polymerization, on the catalyst surface, of HFO generated by the dehydrofluorination reaction or compounds having a triple bond. It is considered that the combination of the change in composition, the change in crystal structure, and the coking phenomenon causes a decrease in catalytic activity.

On the other hand, the present inventors have found that a decrease in the conversion rate of HFC is suppressed by contacting a mixture containing the HFC and the compound A with the catalyst instead of the HFC alone. This is considered to be because the compound A consumes hydrogen fluoride to suppress the action of hydrogen fluoride on the catalyst. For example, when the compound A is the compound (A1) or the compound (A2), hydrogen fluoride is incorporated into the compound A and consumed by the hydrogen fluoride addition reaction of the compound A. When the compound A is the compound (A3), a halogen exchange reaction occurs in which a halogen atom other than a fluorine atom contained in the compound A is exchanged with a fluorine atom of hydrogen fluoride, and the hydrogen fluoride is consumed. In this manner, by the consumption of hydrogen fluoride, a decrease in catalytic activity is suppressed. Specifically, a decrease in catalytic activity with respect to the amount of HFC contacted with the catalyst is suppressed. Accordingly, it is presumed that a decrease in the conversion rate of HFC is suppressed, and the maintenance rate of the HFC conversion rate with respect to the amount of the HFC contacted with the catalyst is increased.

Hereinafter, an embodiment of the method of producing HFO of the disclosure will be described in detail.

### (HFC)

In the method of producing HFO of the present embodiment. HFC is used as a raw material.

HFC is a compound composed of a carbon atom, a hydrogen atom, and a fluorine atom and having no unsaturated bond (for example, a double bond or a triple bond).

A number of carbon atoms in the HFC is, for example, from 2 to 10, and from the viewpoint of easily bringing the HFC into a gas phase state when the HFC is contacted with a catalyst, the number of carbon atoms is preferably from 2 to 8, and more preferably from 2 to 5.

A molecular weight of the HFC is, for example, from 40 to 600, and from the viewpoint of easily bringing the HFC into a gas phase state when the HFC is contacted with the catalyst, the molecular weight is preferably from 40 to 500, and more preferably from 40 to 250.

A boiling point of the HFC is, for example, from −100 to 300° C., and from the viewpoint of easily bringing the HFC into a gas phase state when the HFC is contacted with the catalyst, the boiling point is preferably from −100 to 150° C., and more preferably from −100 to 50° C.

The HFC is preferably a compound represented by the following Formula (1). Hereinafter, the compound represented by Formula (1) is also referred to as “compound (1)”.

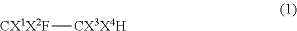

In Formula (1), each of X¹, X², X³, and X⁴ independently represents a hydrogen atom or a fluorine atom, at least one of X¹, X², X³, or X⁴ is a fluorine atom, and at least one of X¹, X², X³, or X⁴ is a hydrogen atom.

Examples of the compound (1) include the following compounds.
CHF₂CH₃: 1,1-Difluoroethane (HFC-152a)
CH₂FCH₂F: 1,2-Difluoroethane (HFC-152)
CF₃CH₃: 1,1,1-Trifluoroethane (HFC-143a)
CHF₂CH₂F: 1,1,2-Trifluoroethane (HFC-143)
CF₃CH₂F: 1,1,1,2-Tetrafluoroethane (HFC-134a)
CHF₂CHF₂: 1,1,2,2-Tetrafluoroethane (HFC-134)

Among them, the HFC is preferably at least one selected from the group consisting of HFC-143a. HFC-143, HFC-134a, and HFC-134 from the viewpoint of reducing side reactions and suppressing generation of byproducts. In addition, since one HFO can be obtained with a high selectivity, the HFC is preferably at least one selected from the group consisting of HFC-143 and HFC-134a, and more preferably HFC-134a.

### (HFO)

In the method of producing HFO of the embodiment, the HFO corresponding to the used HFC is obtained as a reaction product. HFO is a compound composed of carbon atoms, hydrogen atoms, and fluorine atoms, and has a double bond.

A number of carbon atoms in the HFO is the same as the number of carbon atoms in the used HFC, and examples thereof include 2 to 10, and the number of carbon atoms is preferably 2 to 8, and more preferably 2 to 5.

When the compound (1) is used as the HFC, the obtained HFO is a compound represented by the following Formula (2). Hereinafter, the compound represented by Formula (2) is also referred to as “compound (2)”.

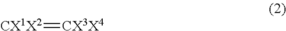

In Formula (2), each of X¹, X², X³, and X⁴ independently represents a hydrogen atom or a fluorine atom, at least one of X¹, X², X³, or X⁴ is a fluorine atom, and at least one of X¹, X², X³, or X⁴ is a hydrogen atom.

Examples of the compound (2) include the following compounds.
CHF=CH₂: Fluoroethylene
CF₂=CH₂: 1,1-Difluoroethylene (HFO-1132a)
CHF═CHF: 1,2-Difluoroethylene (HFO-1132(E), HFO-1132(Z))
CHF═CF₂: Trifluoroethylene (HFO-1123)

Among them, the HFO is preferably at least one selected from the group consisting of HFO-1132, HFO-1132a, and HFO-1123 from the viewpoint of utility as a refrigerant composition.

In the method of producing HFO of the embodiment, as reaction products, olefins other than HFO may be generated together with the HFO corresponding to the used HFC.

Examples of the olefin other than HFO include ethylene.

In the method of producing HFO of the embodiment, it is preferable that the HFC is the compound (1) and the HFO is the compound (2) from the viewpoint that the reaction proceeds more selectively.

In the method of producing HFO of the embodiment, from the viewpoint that the reaction proceeds more selectively, it is more preferable that HFC is at least one selected from the group consisting of HFC-143a, HFC-143, HFC-134a, and HFC-134, and HFO is at least one selected from the group consisting of HFO-1132, HFO-1132a, and HFO-1123.

In particular, in the method of producing HFO of the embodiment, from the viewpoint that the reaction proceeds more selectively, it is preferable that the HFC is HFC-134a and the HFO is HFO-1123, or that the HFC is HFC-143 and the HFO is HFO-1132, and it is more preferable that the HFC is HFC-134a and the HFO is HFO-1123.

### (Compound A)

The compound A is at least one selected from the group consisting of a compound represented by the following Formula (A1), a compound represented by the following Formula (A2), and a compound represented by the following Formula (A3).

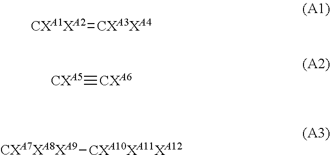

In Formulas (A1) and (A2), each of X^{A1}, X^{A2}, X^{A3}, X^{A4}, X^{A5}, and X^{A6} independently represents a hydrogen atom, a fluorine atom, a chlorine atom, or a hydrocarbon chain having 1 to 8 carbon atoms which may be substituted with at least one of a fluorine atom or a chlorine atom, at least one of X^{A1}, X^{A2}, X^{A3}, or X^{A4} has at least one of a hydrogen atom or a chlorine atom, and at least one of X^{A5} or X^{A6} has at least one of a hydrogen atom or a chlorine atom. At least one of X^{A1}, X^{A2}, X^{A3}, or X^{A4} preferably represents a hydrogen atom or a chlorine atom, and more preferably represents a chlorine atom. At least one of X^{A5} or X^{A6} preferably represents a hydrogen atom or a chlorine atom, and more preferably represents a chlorine atom.

In Formula (A3), each of X^{A7}, X^{A8}, X^{A9}, X^{A10}, X^{A11}, and X^{A12} independently represents a hydrogen atom, a halogen atom, or a hydrocarbon chain having 1 to 8 carbon atoms which may be substituted with a halogen atom, and at least one of X^{A7}, X^{A8}, X^{A9}, X^{A10}, X^{A11}, or X^{A12} has a halogen atom other than a fluorine atom. Examples of the halogen atom include a fluorine atom, a chlorine atom, a bromine atom, and an iodine atom. At least one of X^{A7}, X^{A8}, X^{A9}, X^{A10}, X^{A11}, or X^{A12} preferably represents a halogen atom other than a fluorine atom, and more preferably represents a chlorine atom.

The compound (A1) and the compound (A2) are compounds in which an addition reaction of hydrogen fluoride can occur. Since the compound (A1) and the compound (A2) have an unsaturated bond, an addition reaction of hydrogen fluoride easily occurs. In addition, since the compound (A1) and the compound (A2) have at least one of a hydrogen atom or a chlorine atom, for example, coking caused by a disproportionation reaction on the catalyst is suppressed as compared with a case where X^{A1} to X^{A6} in the formula are all fluorine atoms.

The hydrocarbon chain represented by X^{A1} to X^{A6} in Formulas (A1) and (A2) has 1 to 8 carbon atoms and may be unsubstituted or substituted with at least one of a fluorine atom or a chlorine atom. At least one of a hydrogen atom or a chlorine atom of the compound (A1) and the compound (A2) may be contained in a hydrocarbon chain represented by X^{A1} to X^{A6}.

That is, in the compound (A1), at least one of X^{A1}, X^{A2}, X^{A3}, or X^{A4} is a hydrogen atom, a chlorine atom, an unsubstituted hydrocarbon chain, or a hydrocarbon chain substituted with a chlorine atom, and is preferably a hydrogen atom or a chlorine atom, and more preferably a chlorine atom.

In the compound (A2), at least one of X^{A5} or X^{A6} is a hydrogen atom, a chlorine atom, an unsubstituted hydrocarbon chain, or a hydrocarbon chain substituted with a chlorine atom, preferably a hydrogen atom or a chlorine atom, and more preferably a chlorine atom.

The compound (A3) is a compound capable of undergoing a halogen exchange reaction with hydrogen fluoride. Since the compound (A3) has a halogen atom other than a fluorine atom, a halogen exchange reaction in which a halogen atom other than a fluorine atom of the compound (A3) is exchanged with a fluorine atom of hydrogen fluoride easily occurs. Halogen atoms other than fluorine atoms include chlorine atoms, bromine atoms, and iodine atoms, with chlorine atoms being preferable.

The hydrocarbon chain represented by X^{A7} to X^{A12} in Formula (A3) has 1 to 8 carbon atoms and may be unsubstituted or may be substituted with a halogen atom. The halogen atom other than the fluorine atom of the compound (A3) may be contained in a hydrocarbon chain represented by X^{A7} to X^{A12}.

That is, in the compound (A3), at least one of X^{A7}, X^{A8}, X^{A9}, X^{A10}, X^{A11}, or X^{A12} is a chlorine atom, a bromine atom, an iodine atom, a hydrocarbon chain substituted with a chlorine atom, a hydrocarbon chain substituted with a bromine atom, or a hydrocarbon chain substituted with an iodine atom, preferably a chlorine atom or a hydrocarbon chain substituted with a chlorine atom, and more preferably a chlorine atom.

Examples of the hydrocarbon chain having 1 to 8 carbon atoms include an alkyl group having 1 to 8 carbon atoms, an alkenyl group having 2 to 8 carbon atoms, and an alkynyl group having 2 to 8 carbon atoms.

The alkyl group having 1 to 8 carbon atoms may be either linear or branched, and may be cyclic. Examples of alkyl groups having 1 to 8 carbon atoms include methyl group, ethyl group, propyl group, isopropyl group, butyl group, isobutyl group, s-butyl group, t-butyl group, pentyl group, isopentyl group, neopentyl group, heptyl group, octyl group, 2-methylbutyl group, n-hexyl group, 1-methylpentyl group, 2-methylpentyl group, 3-methylpentyl group, 4-methylpentyl group, 1-ethylbutyl group, 2-ethylbutyl group, 1,1-dimethylbutyl group, 2,2-dimethylbutyl group, 3,3-dimethylbutyl group, 1-ethyl-1-methylpropyl group, cyclopentyl group, cyclohexyl group, cycloheptyl group, and cyclooctyl group.

The alkenyl group having 2 to 8 carbon atoms may be linear or branched, and may also be cyclic. Examples of alkenyl groups having 2 to 8 carbon atoms include vinyl group, allyl group, 1-propenyl group, 2-propenyl group, isopropenyl group, butenyl group, isobutenyl group, pentenyl group, isopentenyl group, hexenyl group, isohexenyl group, heptenyl group, isoheptenyl group, octenyl group, and isooctenyl group.

An alkynyl group having 2 to 8 carbon atoms may be linear or branched, and may also be cyclic. Examples of alkynyl groups having 2 to 8 carbon atoms include ethynyl group, 1-propynyl group, 2-propynyl group, butynyl group, pentynyl group, hexynyl group, heptynyl group, octynyl group, cyclohexynyl group, cycloheptynyl group, and cyclooctynyl group.

A number of carbon atoms in the compound (A1) is, for example, from 2 to 10, from the viewpoint of easily contacting the compound with a catalyst, the number of carbon atoms is preferably from 2 to 8, and more preferably from 2 to 6. A number of carbon atoms in the compound (A2) is, for example, from 2 to 10, and from the viewpoint of easily contacting the compound with a catalyst, the number of carbon atoms is preferably from 2 to 8, and more preferably from 2 to 4. A number of carbon atoms in the compound (A3) is, for example, from 2 to 8, and from the viewpoint of easily contacting the compound with a catalyst, the number of carbon atoms is preferably from 2 to 6, and more preferably from 2 to 4.

A boiling point of the compound (A1) is, for example, from −50 to 200° C., and from the viewpoint of easily contacting the compound with a catalyst, the boiling point is preferably from −50 to 150° C., and more preferably from −50 to 120° C. A boiling point of the compound (A2) is, for example, from −100 to 200° C., and from the viewpoint of easily contacting the compound with a catalyst, the boiling point is preferably from −100 to 150° C., and more preferably from −100 to 120° C. A boiling point of the compound (A3) is, for example, from −50 to 200° C., and from the viewpoint of easily contacting the compound with a catalyst, the boiling point is preferably in a range of from −50 to 150° C., and more preferably from −50 to 120° C.

Examples of the compound A include tetrachloroethane, trichloroethylene, trichloroethane, 1,2-dichloroethylene, and 1,1-dichloroethylene.

From the viewpoint of suppressing a decrease in the conversion rate of HFC, the compound A preferably includes at least one selected from the group consisting of tetrachloroethane, trichloroethylene, trichloroethane, 1,2-dichloroethylene, and 1,1-dichloroethylene, and more preferably includes at least one selected from the group consisting of trichloroethylene, 1,2-dichloroethylene, and 1,1-dichloroethylene.

In the embodiment, it is preferable that the HFC is the compound (1), the HFO is the compound (2), and the compound A includes a compound represented by the following Formula (B1).

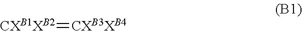

In Formula (B1), each of X^{B1}, X^{B2}, X^{B3}, and X^{B4} independently represents a group corresponding to X¹, X², X³, and X⁴ in Formulas (1) and (2),
in a case in which each of X¹, X², X³, and X⁴ in Formulas (1) and (2) independently represents a hydrogen atom, each of X^{B1}, X^{B2}, X^{B3}, and X^{B4} is a hydrogen atom, and in a case in which each of X, X², X³, and X⁴ in Formulas (1) and (2) independently represents a fluorine atom, each of X^{B1}, X^{B2}, X^{B3}, and X^{B4} is a chlorine atom.

Hereinafter, a compound represented by Formula (B1) is also referred to as “compound (B1)”.

Specifically, for example, when the HFC is HFC-134a and the HFO is HFO-1123, the compound (B1) includes trichloroethylene. For example, when the HFC is HFC-143 and the HFO is HFO-1132, the compound (B1) includes 1,2-dichloroethylene.

The compound (B1) only needs to be contained in the mixture when the mixture is contacted with the catalyst, and a mixture containing the compound (B1) generated by the reaction of the precursor of the compound (B1) may be contacted with the catalyst. Specifically, for example, when HFC-143 is used as the HFC and 1,2-dichloroethylene is used as the compound (B1), a mixture containing HFC-143 and 1,1,2-trichloroethane as a precursor of 1,2-dichloroethylene may be prepared, and a mixture obtained by generating 1,2-dichloroethylene by a dehydrochlorination reaction of 1,1,2-trichloroethane may be contacted with the catalyst.

When the compound A contains the compound (B1), hydrogen fluoride generated by the dehydrofluorination reaction of HFC reacts with the compound (B1), and HFC as a raw material is easily obtained from the reaction product. When the reaction product of the compound (B1) and hydrogen fluoride is further reacted to easily obtain HFC as a raw material, HFO as a target product can be obtained by the dehydrofluorination reaction of the obtained HFC, and the compound (B1) can be effectively used.

In the embodiment, from the viewpoint of suppressing a decrease in the conversion rate of HFC and from the viewpoint of easily obtaining HFC from the reaction product of the compound A, it is preferable that the HFC is at least one selected from the group consisting of HFC-134 and HFC-134a, and that the compound A is trichloroethylene.

In the mixture containing the HFC and the compound A, a molar ratio of the compound A to the HFC is preferably from 0.05 to 0.99 from the viewpoint of suppressing a decrease in the conversion rate of HFC.

When the molar ratio of the compound A to the HFC is equal to or greater than the above lower limit value, the consumption of hydrogen fluoride generated by the dehydrofluorination reaction of the HFC is more likely to occur, and a decrease in the conversion rate of HFC is suppressed. From that viewpoint, the molar ratio of the compound A to the HFC is preferably 0.1 or more, more preferably 0.2 or more, and still more preferably 0.3 or more.

When the molar ratio of the compound A to the HFC is equal to or less than the above upper limit value, inhibition of the progress of the dehydrofluorination reaction due to an excessive amount of the compound A is suppressed, and a decrease in the conversion rate of HFC is suppressed. From that viewpoint, the molar ratio of the compound A to the HFC is preferably 0.9 or less, more preferably 0.8 or less, and still more preferably 0.7 or less.

### (Other Components)

The mixture containing the HFC and the compound A may contain other components as necessary.

Other components include, for example, isomers, disproportionation products, and impurities obtained during the production of HFC, as well as inert gases.

Examples of inert gases include nitrogen, helium, argon, octafluorocyclobutane, and carbon dioxide, with nitrogen being preferable. When the mixture contains an inert gas, a molar ratio of the HFC to the inert gas is, for example, from 0.1 to 30, and may also be from 0.5 to 25.

In the embodiment, since the hydrogen fluoride generated by the dehydrofluorination reaction of HFC is consumed by the compound A, the mixture need not contain an inert gas. A total content of the HFC and the compound A in the mixture may be 80 mol % or more, 90 mol % or more, or 95 mol % or more with respect to the entire mixture.

### (Catalyst)

In the method of producing HFO of the embodiment, a mixture containing the HFC and the compound A is contacted with a catalyst.

Examples of the catalyst include oxides, hydroxides, halides, oxyhalides, and inorganic salts of chromium, aluminum, copper, zinc, zirconium, iron, nickel, and magnesium, and mixtures thereof, but are not limited thereto. Any of these may be halogenated, as appropriate.

From the viewpoint of the conversion rate of HFC, among these, preferable examples of the catalyst include Al₂O₃, Cr₂O₃, Cr₂O₃/Al₂O₃, Cr₂O₃/AlF₃, NiCl₂/Cr₂O₃/Al₂O₃, NiCl₂/AlF₃, and mixtures thereof, and more preferably, Al₂O₃.

Examples of Al₂O₃ include γ-alumina, θ-alumina, α-alumina, β-alumina, η-alumina, boehmite, and gibbsite, which have different crystal structures, and from the viewpoint of suppressing a decrease in the conversion rate of HFC, α-alumina is preferable.

The crystal structure of alumina contained in the catalyst can be confirmed by a diffraction pattern obtained by an X-ray diffraction method, in other words, an X-ray diffractometer (XRD). As the XRD, a commercially available apparatus can be used, and for example, “SmartLab” manufactured by Rigaku Corporation is used. When there are peaks of d=26.62, 35.21, 37.85, 43.43, 52.65, and 57.61 Å in the diffraction pattern, it can be determined that α-alumina is contained. This analysis is to be conducted on the catalyst immediately before being contacted with the mixture containing the HFC and the compound A, or on a catalyst that reproduces the same state as the catalyst immediately before being contacted with the mixture containing the HFC and the compound A.

When the catalyst contains α-alumina, a content of the α-alumina crystal structure in the catalyst may be 65 mass % or more, 70 mass % or more, 75 mass % or more, 80 mass % or more, 85 mass % or more, or even 100 mass %.

The content of the α-alumina crystal structure in the catalyst can be confirmed by performing Rietveld analysis based on the crystal structure obtained from XRD. Specifically, a peak obtained by performing XRD measurement on the catalyst is compared with a known peak model derived from each alumina structure, and Rietveld analysis is performed to calculate the mass ratio between each crystal structure.

The form of the catalyst is not particularly limited, and may be a powder, a pellet shape, or a spherical shape.

The catalyst is preferably a spherical or pellet-shaped molded body from the viewpoint of handling because this provides excellent packing properties when packed into a reactor and excellent flowability of the reaction gas.

Unlike the powder, the molded body is obtained by, for example, compression-molding the powder in a mold.

A specific surface area of the catalyst is, for example, from 0.1 to 500 m²/g, and from the viewpoint of the reaction efficiency in the dehydrofluorination reaction of HFC and the action efficiency of hydrogen fluoride and the compound A, the specific surface area is preferably from 0.1 to 100 m²/g, and more preferably 1 to 50 m²/g. The specific surface area is a value measured by a BET method (BET specific surface area).

A bulk density of the catalyst is, for example, from 0.4 to 1.5 g/mL, and from the viewpoint of production efficiency and reaction efficiency of HFO, the bulk density is preferably from 0.5 to 1.4 g/mL, more preferably from 0.6 to 1.3 g/mL, and still more preferably 0.7 to 1.2 g/mL.

### (Reaction Conditions)

The production of the fluoroolefin in the embodiment may be performed in a gas phase or may be in a liquid phase. From the viewpoint of enabling production by a highly versatile reaction facility, it is preferable that the HFC is contacted with the catalyst in a gas phase state.

The reactor that contacts the mixture with the catalyst may be any reactor that can withstand the temperature and pressure described later, and the shape and structure are not particularly limited. Examples of the reactor include a cylindrical vertical reactor. Examples of the material of the reactor include glass, stainless steel, iron, nickel, and alloys mainly composed of iron or nickel. The reactor may include heating means such as an electric heater that heats the inside of the reactor.

The catalyst may be stored in any form of a fixed bed type, a fluidized bed type, or a movable bed type. In the case of the fixed bed type, the form may be either a horizontal fixed bed type or a vertical fixed bed type.

The reaction type may be a flow type or a batch type.

In a fixed-bed reactor, various molded bodies of catalyst-supported carriers are packed in order to reduce the pressure loss of the reaction fluid. Similar to a fixed-bed reactor, a system in which the catalyst is packed, moved by gravity, and withdrawn from the bottom of the reactor for regeneration is referred to as a moving bed. In the fluidized bed reactor, the catalyst particles are suspended in the reaction fluid and move in the reactor because the operation is performed such that the catalyst bed exhibits fluid-like characteristics by the reaction fluid. A fixed-bed reactor is preferable because this offers a wide range of options for the catalyst shape and allows suppression of catalyst wear. As fixed-bed reactors, there are tubular reactors and tank-type reactors, and tubular reactors are preferable due to the ease of controlling the reaction temperature. Further, a multi-tube heat exchange type reaction in which a large number of reaction tubes having a small tube diameter are arranged in parallel and a heat medium is circulated outside can be employed. When plural reactors are provided in series, plural catalyst beds are provided. The catalyst bed may have at least one stage, and may have two or more stages.

From the viewpoint of improving the conversion rate of HFC, in the embodiment, it is preferable to perform the process in a flow-type system using a fixed-bed reactor (particularly a vertical fixed-bed type reactor).

In the embodiment, it is preferable to contact the mixture with the catalyst at a temperature from 100 to 800° C., it is more preferable to contact the mixture with the catalyst at a temperature from 300 to 800° C., it is still more preferable to contact the mixture with the catalyst at a temperature from 400 to 700° C., and it is particularly preferable to contact the mixture with the catalyst at a temperature from 400 to 600° C. When the contact temperature is 100° C. or higher, the dehydrofluorination reaction of HFC appropriately proceeds, and the conversion rate of HFC is improved. On the other hand, when the contact temperature is 800° C. or lower, a decrease in selectivity due to carbon-carbon bond cleavage of HFC and a disproportionation reaction of a product (unsaturated compound) can be suppressed.

Since the dehydrofluorination reaction is generally an endothermic reaction, a decrease in the conversion rate of HFC can be suppressed by appropriately maintaining the reaction temperature. When the reaction temperature in the catalyst bed increases, the conversion rate of the raw material increases. Therefore, it is preferable to keep the reaction temperature in the catalyst bed at a desired temperature such that a high conversion rate of HFC can be maintained. In order to maintain the reaction temperature in the catalyst bed at a desired temperature, for example, a method of externally heating the catalyst bed with a heat medium or the like can be mentioned. The catalyst usually deteriorates over time as the reaction proceeds. Even when the conversion of the raw material decreases due to catalyst deterioration, the catalyst bed is heated with a heating medium or the like to appropriately maintain or increase the reaction temperature, and accordingly, a decrease in the conversion rate of HFC can be suppressed. When the temperature of the catalyst bed is maintained or increased, the temperature increase width is preferably 50° C. or less in order to suppress rapid deterioration of the catalyst.

The reaction zone starts from the gas inlet portion of the raw material gas as a mixture at the beginning of the reaction. When the catalyst in the gas inlet portion of the raw material gas deteriorates over time as the reaction proceeds, the reaction zone moves to the downstream side in the gas flow direction. In the vicinity of the downstream side of the reaction zone, lower-temperature product gas generated in the reaction zone flows in, and therefore, the vicinity of the downstream side is typically the lowest in temperature within the catalyst bed. In the embodiment, the temperature of the lowest temperature region of the catalyst bed is referred to as “the lowest temperature of the catalyst bed”. The temperature from the vicinity of the downstream side to the further downstream side is usually higher than the lowest temperature of the catalyst bed as the distance from the reaction zone increases.

In the embodiment, the raw material gas as a mixture containing the HFC and the compound A may be supplied to the reactor at normal temperature. However, it is preferable that the raw material gas is appropriately heated (preheated) before being supplied to the reactor. When preheating is performed, the raw material gas is preferably heated to a temperature from 80 to 600° C. and then supplied to the reactor. When preheating is performed to 80° C. or higher, the internal temperature of the reactor is less likely to decrease, and the set conversion rate of HFC is easily achieved. In addition, when preheating is performed to 600° C. or lower, the internal temperature of the reactor is unlikely to increase, an undesirable reaction is suppressed, and the selectivity is improved.

Since the dehydrofluorination reaction in the embodiment is a reaction in which the number of molecules increases, the forward reaction becomes less favorable as the pressure increases.

A pressure when the mixture containing HFC and the compound A is contacted with the catalyst is not particularly limited, but from the viewpoint of improving the conversion rate of HFC, the pressure is preferably from −0.05 to 2 MPa, more preferably from −0.01 to 1 MPa, and still more preferably from normal pressure to 0.5 MPa.

In the disclosure, pressure means gauge pressure.

A contact time between the mixture and the catalyst is preferably from 0.1 to 100.0 seconds, more preferably from 0.5 to 100.0 seconds, still more preferably from 1.0 to 50.0 seconds, and particularly preferably from 1.0 to 20.0 seconds.

The contact time (seconds) is calculated using the following formula.

$$Contact time ( sec ) = [ Length of reactor packed with catalyst ( cm ) ] / [ Linear velocity ( cm / sec ) ]$$

Linear velocity means the rate at which the mixture passes through the catalyst per unit time.

A contact time (g·sec/mL) between the HFC contained in the mixture and the catalyst is preferably from 1 to 200 g·sec/mL, more preferably from 5 to 175 g sec/mL, still more preferably from 7 to 150 g·sec/mL, and particularly preferably from 10 to 125 g·sec/mL. When the contact time (g·sec/mL) is 1 g·sec/mL or more, the conversion rate of HFC is improved. When the contact time (g sec/mL) is 200 g sec/mL or less, the equipment cost can be suppressed.

The contact time (g·sec/mL) is calculated using the following formula.

$$Contact time ( g \cdot sec / mL ) = [ Packed amount of catalyst ( g ) ] / [ Flow amount of HFC ( mL / sec ) ]$$

From the viewpoint of further suppressing a decrease in conversion rate of HFC, it is preferable that the mixture and the catalyst are contacted with each other in a gas phase in the presence of water, and a concentration of water is less than 500 ppm with respect to the total amount of the raw material gas as a mixture.

In the embodiment, it is considered that the reaction proceeds by the Lewis acid sites on the surface of the catalyst serving as active sites. By performing the dehydrofluorination reaction in the gas phase in the presence of water, water is adsorbed to the Lewis acid sites on the surface of the catalyst. It is presumed that by setting the concentration of water to less than 500 ppm with respect to the total amount of the raw material gas as a mixture, the Lewis acid sites on the surface of the catalyst are crushed to form a structure similar to the Bronsted acid sites, and a decrease in the activity of the catalyst is suppressed.

As a general method for measuring the concentration of water, a method using a commercially available Karl Fischer moisture analyzer can be mentioned. When the concentration of water is less than 500 ppm with respect to the total amount of the mixture, the conversion rate of HFC is high, and a target product is obtained with high selectivity. The concentration of water is preferably 300 ppm or less, more preferably 100 ppm or less, still more preferably 50 ppm or less, and particularly preferably 10 ppm or less from the viewpoint of further improving the conversion rate and obtaining the target compound with a higher selectivity. The concentration of water is preferably low, but is preferably 0.5 ppm or more, and more preferably 1 ppm or more from the viewpoint of reducing the cost of the dehydration treatment of the HFC and facilitating the process management.

The concentration of water is the content of water contained in the raw material gas when the raw material gas as a mixture is contacted with the catalyst. The concentration of water may be replaced with the content of water contained in the raw material gas before flowing into the reactor.

In the embodiment, it is preferable to further include a step of drying the catalyst before contacting the raw material gas as a mixture with the catalyst. By drying the catalyst, the water contained in the catalyst is removed, the reactivity to HFC is enhanced, and the conversion rate of HFC is improved.

The method for drying the catalyst is not particularly limited, and the catalyst may be dried before being packed into the reactor or may be dried after being packed into the reactor. When the catalyst is packed into the reactor and then dried, the drying of the catalyst simultaneously enables preheating of the reactor, which is preferable. Specifically, it is preferable to dry the catalyst by packing the reactor with the catalyst and heating the reactor while circulating an inert gas.

The method of producing HFO of the embodiment may be performed in the presence of an oxidizing agent. The oxidizing agent is preferably oxygen, chlorine, bromine, or iodine from the viewpoint of having a high conversion rate and obtaining the target compound with a high selectivity. Among them, oxygen is more preferable. In the disclosure, a concentration of the oxidizing agent is preferably from 0.01 to 21 mol % with respect to the raw material gas. The concentration of the oxidizing agent is more preferably from 1 to 20 mol %, still more preferably from 5 to 18 mol %, and particularly preferably from 7.5 to 16 mol % with respect to the raw material compound from the viewpoint of further improving the conversion rate and obtaining the target compound with a higher selectivity.

In the disclosure, the conversion rate means the ratio (mol %) of the total molar amount of the reaction product of the raw material compound contained in the outflow gas from the reactor outlet to the molar amount of the raw material compound supplied to the reactor.

Specifically, examples of the conversion rate include a conversion rate when one hour has elapsed after the raw material gas as a mixture is contacted with the catalyst. The conversion rate of HFC after one hour has elapsed is preferably 2% or more, more preferably 3% or more, particularly preferably 5% or more, and most preferably 7% or more from the viewpoint of productivity. The conversion rate of HFC after one hour has elapsed may be 30% or less, 25% or less, 20% or less, 15% or less, or 13% or less from the viewpoint of maintaining the activity of the catalyst.

In the disclosure, the selectivity means the ratio (mol %) of the molar amount of the target product contained in the reactor outlet gas to the total molar amount of the reaction products of the raw material compound contained in the reactor outlet gas.

Specifically, the selectivity includes, for example, a selectivity after one hour has elapsed from the time when the raw material gas as a mixture is contacted with the catalyst.

The selectivity is preferably 100% because a purification step after the reaction is unnecessary, but a side reaction may also occur in a reaction temperature region for obtaining a desired conversion rate. The selectivity is preferably high because the amount of waste can be reduced, the energy load of the purification step after the reaction can be reduced, and the catalyst life can be prolonged. The selectivity of HFO after one hour has elapsed is preferably 90% or more, more preferably 93% or more, still more preferably 95% or more.

In particular, in the present embodiment, by using the HFC-134a as the HFC, the HFO-1123 can be obtained with a high selectivity.

When HFC-134a is used as the HFC and the target product is HFO-1123, examples of the compound other than the target product among the reaction products of the HFC which is the raw material compound contained in the reactor outlet gas include HFC-134, 1,1-difluoroethylene (VdF), FJZ-1,2-difluoroethylene (E/Z-HFO-1132), hydrogen fluoride, carbon monoxide, carbon dioxide, and water.

The method of producing HFO in the embodiment may include a step of generating HFO and hydrogen fluoride by the dehydrofluorination reaction of HFC, and a step of allowing the compound A to act on the hydrogen fluoride generated by the dehydrofluorination reaction of HFC. In the reaction field of the dehydrofluorination reaction of HFC, the compound A acts on hydrogen fluoride generated by the dehydrofluorination reaction, whereby hydrogen fluoride is consumed, and the amount of hydrogen fluoride present in the reaction field decreases. The step of generating HFO and hydrogen fluoride by the dehydrofluorination reaction of HFC and the step of allowing the compound A to act on hydrogen fluoride generated by the dehydrofluorination reaction of HFC preferably proceed in the same reactor, and preferably proceed in a temporally overlapping manner.

In the step in which the compound A acts on hydrogen fluoride, a chemical reaction between hydrogen fluoride and the compound A may occur. For example, when the compound A is the compound (A1) or the compound (A2), a hydrogen fluoride addition reaction in which hydrogen fluoride is added to the compound A may occur. For example, in the case where the compound A is the compound (A3), a halogen exchange reaction in which a halogen atom other than a fluorine atom possessed by the compound A is exchanged with a fluorine atom of hydrogen fluoride may occur.

The conversion rate of the compound A after one hour has elapsed in the chemical reaction between hydrogen fluoride and the compound A is, for example, 1% or more, may be 5% or more, or may be 10% or more.

In the step in which the compound A acts on hydrogen fluoride, not only the chemical reaction between hydrogen fluoride and the compound A but also adsorption of hydrogen fluoride to the compound A may occur, and the compound A may inhibit contact of hydrogen fluoride with the catalyst.

According to the embodiment, even when a large amount of raw material (for example, 1.2 moles or more of HFC) is contacted with the catalyst, a decrease in the conversion rate is suppressed. A ratio of the conversion rate after 1.2 mol of HFC is contacted with the catalyst to the conversion rate at the initial stage (for example, when 0.04 mol of HFC is contacted with the catalyst), that is, a conversion rate retention ratio is preferably 80% or more, more preferably 85% or more, and still more preferably 90% or more.

### EXAMPLES

Hereinafter, the disclosure will be described more specifically with reference to examples, but the disclosure is not limited to the following examples unless it goes beyond the gist of the present disclosure.

### Example 1

0.88 g of α-alumina (product name: “FGL-40”, manufactured by Iwatani Chemical Industry Co., Ltd., specific surface area: 2.7 m²/g, bulk density: 1.0 g/ml, content of α-alumina crystal structure: 65 mass % or more) was weighed and used as the catalyst. A stainless steel (SUS304) reaction tube having an inner diameter of 1.02 cm and a length of 30 cm was packed with a catalyst such that the packed bed volume was 1.0 cm³ (packed bed height: 1.3 cm), and the tube was placed in a tubular electric furnace. While flowing nitrogen, the catalyst-packed portion was heated to 475° C. in the tubular furnace to dehydrate the catalyst.

Thereafter, a dehydrofluorination reaction to HFO-1123 was performed for 15 hours under atmospheric pressure by flowing a mixed gas 1 of trichloroethylene/HFC-134a at a molar ratio of 0.5/1 with a contact time of 1.0 second through the catalyst at a temperature of 450° C.

Subsequently, a dehydrofluorination reaction to HFO-1123 was performed for 14 hours under atmospheric pressure by flowing a mixed gas 2 of trichloroethane/HFC-134a at a molar ratio of 0.4/1 with a contact time of 1.0 second through the catalyst at a temperature of 500° C., instead of the mixed gas 1.

In the flow of the mixed gas 1 and the mixed gas 2, the flow amount of HFC-134a was 15.0 Nml/min in both cases, and the amount (that is, the raw material load) of HFC-134a flowed per 1 m³ of the catalyst per hour was 4,099 kg/hr/m³ in both cases. The total flow amount of HFC-134a in the flow of the mixed gas 1 and the mixed gas 2 for a total of 29 hours was 1.2 mol.

The concentration of water in each of the mixed gas 1 and the mixed gas 2 was measured using a Karl Fischer moisture analyzer and found to be less than 500 ppm in both cases.

After 1 hour from the start of the reaction and after the end of the reaction for a total of 29 hours, the product gas (hereinafter, also referred to as “reactor outlet gas”) taken out from the outlet of the reactor was analyzed by gas chromatography. Specifically, analysis was performed by installing a column (product name: “DB-1301”, manufactured by Agilent, length: 60 m, inner diameter: 0.25 mm, film thickness: 1 μm) on a gas chromatograph (product name: “GC6850”, manufactured by Agilent). Using the molar amounts calculated from the area percentage (GC Area %) of the reactor outlet gas, the conversion rate of HFC-134a, the selectivity of HFO-1123, the conversion rates of trichloroethylene and trichloroethane used as the compound A, and the selectivities of the hydrogen fluoride adduct of trichloroethylene and the halogen exchange reaction product of trichloroethane were calculated.

### (Conversion Rate of HFC-134a)

The ratio (mol %) of the total molar amount M1 of the reaction product of HFC-134a contained in the reactor outlet gas to the molar amount M134a of HFC-134a supplied to the reactor was taken as the conversion rate of HFC-134a. The total molar amount M1 of the reaction product of HFC-134a was calculated by subtracting the molar amount M2 of HFC-134a contained in the reactor outlet gas from the molar amount M₁₃₄ₐ of HFC-134a supplied to the reactor. The conversion rate retention ratio was calculated by dividing the conversion rate after the end of the reaction by the conversion rate after one hour. The results are shown in Table 1.

$$Conversion rate of HFC - 134 a ( \% ) = ( M 1 / {M}_{134 a} ) \times 100$$

$$M 1 = {M}_{134 a} - M 2$$

### (Selectivity of HFO-1123)

The ratio (mol %) of the molar amount M₁₁₂₃ of HFO-1123 contained in the reactor outlet gas to the total molar amount M1 of the reaction products of HFC-134a contained in the reactor outlet gas was defined as the selectivity of HFO-1123. The results are shown in Table 1.

$$Selectivity of HFO - 1123 ( \% ) = ( {M}_{1123} / M 1 ) \times 100$$

### (Conversion Rate of Compound A)

The ratio (mol %) of the total molar amount M3 of the reaction products of the compound A contained in the reactor outlet gas to the molar amount M_{A} of the compound A supplied to the reactor was defined as the conversion rate of the compound A. The total molar amount M3 of the reaction products of the compound A was calculated by subtracting the molar amount M4 of the compound A contained in the reactor outlet gas from the molar amount M_{A} of the compound A supplied to the reactor. As a result, the conversion rate of the compound A was 98.9% after 1 hour from the start of the reaction, and the conversion rate of the compound A was 100% after the end of the reaction.

$$Conversion rate of compound A ( \% ) = ( M 3 / {M}_{A} ) \times 100$$

$$M 3 = {M}_{A} - M 4$$

### (Selectivity of Hydrogen Fluoride Adduct or Halogen Exchange Reaction Product of Compound A)

The ratio (mol %) of the molar amount M_{B} of the hydrogen fluoride adduct or the halogen exchange reaction product of the compound A contained in the reactor outlet gas to the total molar amount M3 of the reaction product of the compound A contained in the reactor outlet gas was taken as the selectivity of the hydrogen fluoride adduct or the halogen exchange reaction product of the compound A. As a result, the selectivity of the hydrogen fluoride adduct of trichloroethylene as the compound A after 1 hour from the start of the reaction was 0.47%, and the selectivity of the halogen exchange reaction product of trichloroethane as the compound A after completion of the reaction was 11.6%.

Selectivity of hydrogen fluoride adduct or halogen exchange reaction product of compound A (%)=(M_{B}/M3)×100

After the end of the reaction for a total of 29 hours, the generation amount of hydrogen fluoride was calculated from the generation amount of HFO-1123. The results are shown in Table 1 (“HF molar amount” in Table 1).

The catalyst after the end of the reaction for a total of 29 hours was subjected to qualitative and quantitative analysis measurement under the conditions of an X-ray output of 50 kV and 72 mA, a measurement area of 20 mmφ, and a measurement time of 30 minutes using an XRF analyzer (X-ray fluorescence spectrometer, for example, a scanning X-ray fluorescence spectrometer manufactured by Rigaku Corporation, ZSX Primus II), thereby calculating the fluorination ratio of the catalyst per 1 g of generated hydrogen fluoride.

Specifically, the amount of atoms in all the elements present in the measurement region was measured, the amount of atoms in each element was converted into a mass, the percentage of the mass of each element with respect to the mass of all the elements present in the measurement region was calculated from these results, and the fluorination ratio of the catalyst per 1 g of the generated hydrogen fluoride was determined.

The results are shown in Table 1 (“Catalyst fluorination ratio” in Table 1).

The amount of carbon deposition on the catalyst by the coking phenomenon was calculated by measuring the catalyst after the end of the reaction for a total of 29 hours in the same manner as the fluorination ratio of the catalyst. The results are shown in Table 1 (“Catalyst coke amount” in Table 1). The carbon deposition amount per 1 g of the generation amount of HFO-1123 was calculated by dividing the amount of carbon deposition by the generation amount of HFO-1123. The results are shown in Table 1 (“Catalyst coke deposition ratio” in Table 1).

### Example 2

The same catalyst as in Example 1 was installed in the reaction tube, and the catalyst was dehydrated in the same manner as in Example 1.

Thereafter, a dehydrofluorination reaction to HFO-1123 was performed for 95 hours under atmospheric pressure by flowing single gas 3 of HFC-134a with a contact time of 3.9 seconds through the catalyst at a temperature of 450° C.

In the flow of the single gas 3, the flow amount of HFC-134a was 4.6 Nml/min, and the amount of HFC-134a flowed per 1 m³ of the catalyst per hour (that is, the raw material load) was 1,257 kg/hr/m³. The total flow amount of HFC-134a during the flow of the single gas 3 for a total of 95 hours was 1.2 mol.

The concentration of water of the single gas 3 was measured using a Karl Fischer moisture analyzer and found to be less than 5.5 ppm.

In the same manner as in Example 1, the generation amount of hydrogen fluoride (HF molar amount), the fluorination ratio of the catalyst per 1 g of hydrogen fluoride (catalyst fluorination ratio), the amount of carbon deposition (catalyst coke amount), the amount of carbon deposition per 1 g of generation amount of HFO-1123 (catalyst coke deposition ratio), the conversion rate of HFC-134a, the conversion rate retention ratio of HFC-134a, and the selectivity of HFO-1123 were determined. The results are shown in Table 1.

### Example 3

γ-Alumina (product name: “N612N”, manufactured by JGC Catalysts and Chemicals Ltd., specific surface area: 190 m²/g, bulk density: 0.70 g/ml) was used as the catalyst. The catalyst was installed in a reaction tube in the same manner as in Example 1 to dehydrate the catalyst.

Thereafter, a dehydrofluorination reaction to HFO-1123 was performed for 49 hours under atmospheric pressure by flowing mixed gas 4 of nitrogen/HFC-134a at a molar ratio of 0.25/1 with a contact time of 4.5 seconds through the catalyst at a temperature of 450° C.

In the flow of the mixed gas 4, the flow amount of HFC-134a was 4.6 Nml/min in all cases, and the amount of HFC-134a flowed per 1 m³ of the catalyst per hour (that is, the raw material load) was 1,257 kg/hr/m³. The total flow amount of HFC-134a during the flow of the mixed gas 4 for a total of 49 hours was 0.60 mol.

The concentration of water of single gas 4 was measured using a Karl Fischer moisture analyzer and found to be 6.9 ppm.

In the same manner as in Example 1, the generation amount of hydrogen fluoride (HF molar amount), the fluorination ratio of the catalyst per 1 g of hydrogen fluoride (catalyst fluorination ratio), the amount of carbon deposition (catalyst coke amount), the amount of carbon deposition per 1 g of generation amount of HFO-1123 (catalyst coke deposition ratio), the conversion rate of HFC-134a, the conversion rate retention ratio of HFC-134a, and the selectivity of HFO-1123 were determined. The results are shown in Table 1.

TABLE 1

| | | Example 1 | Example 2 | Example 3 |
|---|---|---|---|---|
| Conditions | Catalyst type | α-Alumina | α-Alumina | γ-Alumina |
| | Raw material gas | Trichloroethylene/HFC-134a | HFC-134a | Nitrogen/HFC-134a |
| | | Trichloroethane/HFC-134a | | |
| | Total flow amount of | 1.2 | 1.2 | 0.60 |
| | HFC-134a (mol) | | | |
| Catalyst | HF molar amount (mol) | 0.13 | 0.050 | 0.046 |
| | Catalyst fluorination | 0 | 0.46 | 0.84 |
| | ratio (g/HF-g) | | | |
| | Catalyst coke amount (g) | 0.0099 | 0.0450 | 0.1500 |
| | Catalyst coke deposition | 0.00018 | 0.00900 | 0.03900 |
| | ratio (g/1123-g) | | | |
| Reaction | HFC-134a conversion | 8.9 | 7.9 | 23 |
| | rate after 1 hour (%) | | | |
| | HFC-134a conversion | 11 | 6.1 | 5.8 |
| | rate after completion of | | | |
| | reaction (%) | | | |
| | HFC-134a conversion | 125 | 77 | 26 |
| | rate retention ratio (%) | | | |
| | HFO-1123 selectivity | 100 | 95.1 | 96.2 |
| | after 1 hour (%) | | | |
| | HFO-1123 selectivity | 92.3 | 98.5 | 99.4 |
| | after completion of | | | |
| | reaction (%) | | | |

Example 1 is an example according to the present disclosure, and Examples 2 and 3 are comparative examples.

As shown in Table 1, in Example 1, it was found that by contacting a mixture of HFC and the compound A with the catalyst, the conversion rate retention ratio of HFC is high even during long-term production, and a decrease in the conversion rate was suppressed.

In Example 1, compared to Examples 2 and 3, the fluorination ratio of the catalyst per 1 g of hydrogen fluoride (catalyst fluorination ratio) was lower, and the amount of carbon deposition (catalyst coke amount) as well as the amount of carbon deposition per 1 g of generation amount of HFO-1123 (catalyst coke deposition ratio) were also lower. From this, in Example 1, it is presumed that the suppression of the fluorination of the catalyst and the deposition of carbon contributes to the improvement of the conversion rate retention ratio.

In Example 1, the mixed gas 1 of trichloroethylene/HFC-134a was flowed for 15 hours, and then the mixed gas 2 of trichloroethane/HFC-134a was flowed for 14 hours. If the fluorination of the catalyst and the coking phenomenon occur during the flow of the mixed gas 1, the fluorination amount and the amount of carbon deposition do not theoretically decrease during the flow of the mixed gas 2. Therefore, from the results of Example 1, it is presumed that both trichloroethylene and trichloroethane have an effect of suppressing the fluorination of the catalyst and the deposition of carbon.

The disclosure of Japanese Patent Application No. 2023-089279 filed on May 30, 2023 is incorporated herein by reference in its entirety. In addition, all documents, patent applications, and technical standards described in this specification are incorporated herein by reference to the same extent as if each document, patent application, and technical standard were specifically and individually described to be incorporated by reference.

1. A method of producing a hydrofluoroolefin, the method comprising:
contacting a mixture comprising a hydrofluorocarbon and a compound A, which is at least one selected from the group consisting of a compound represented by the following Formula (A1), a compound represented by the following Formula (A2), and a compound represented by the following Formula (A3), with a catalyst, and
obtaining the hydrofluoroolefin by a dehydrofluorination reaction of the hydrofluorocarbon: 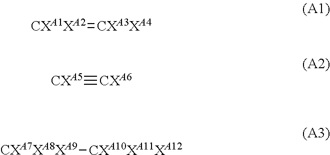
wherein, in Formulas (A1) and (A2), each of X^{A1}, X^{A2}, X^{A3}, X^{A4}, X^{A5}, and X^{A6} independently represents a hydrogen atom, a fluorine atom, a chlorine atom, or a hydrocarbon chain having 1 to 8 carbon atoms which may be substituted with at least one of a fluorine atom or a chlorine atom, at least one of X^{A1}, X^{A2}, X^{A3}, or X^{A4} has at least one of a hydrogen atom or a chlorine atom, and at least one of X^{A5} or X^{A6} has at least one of a hydrogen atom or a chlorine atom, and
in Formula (A3), each of X^{A7}, X^{A8}, X^{A9}, X^{A10}, X^{A11}, and X^{A12} independently represents a hydrogen atom, a halogen atom, or a hydrocarbon chain having 1 to 8 carbon atoms which may be substituted with a halogen atom, and at least one of X^{A7}, X^{A8}, X^{A9}, X^{A10}, X^{A11}, or X^{A12} has a halogen atom other than a fluorine atom.

2. The method of producing a hydrofluoroolefin according to claim 1, wherein the compound A comprises at least one selected from the group consisting of tetrachloroethane, trichloroethylene, trichloroethane, 1,2-dichloroethylene, and 1,1-dichloroethylene.

3. The method of producing a hydrofluoroolefin according to claim 1, wherein the hydrofluorocarbon is a compound represented by the following Formula (1), and the hydrofluoroolefin is a compound represented by the following Formula (2), 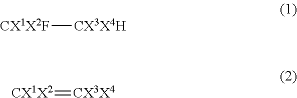
wherein, in Formulas (1) and (2), each of X¹, X², X³, and X⁴ independently represents a hydrogen atom or a fluorine atom, at least one of X¹, X², X³, or X⁴ is a fluorine atom, and at least one of X¹, X², X³, or X⁴ is a hydrogen atom.

4. The method of producing a hydrofluoroolefin according to claim 3, wherein the hydrofluorocarbon is at least one selected from the group consisting of 1,1,1-trifluoroethane, 1,1,2-trifluoroethane, 1,1,2,2-tetrafluoroethane, and 1,1,1,2-tetrafluoroethane, and the hydrofluoroolefin is at least one selected from the group consisting of 1,2-difluoroethylene, 1,1-difluoroethylene, and trifluoroethylene.

5. The method of producing a hydrofluoroolefin according to claim 3, wherein the compound A comprises a compound represented by the following Formula (B1), 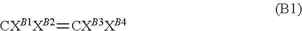
wherein, in Formula (B1), each of X^{B1}, X^{B2}, X^{B3}, and X^{B4} independently represents a group corresponding to X¹, X², X³, and X⁴ in Formulas (1) and (2), in a case in which each of X¹, X², X³, and X⁴ in Formulas (1) and (2) independently represents a hydrogen atom, each of X^{B1}, X^{B2}, X^{B3}, and X^{B4} is a hydrogen atom, and in a case in which each of X¹, X², X³, and X⁴ in Formulas (1) and (2) independently represents a fluorine atom, each of X^{B1}, X^{B2}, X^{B3}, and X^{B4} is a chlorine atom.

6. The method of producing a hydrofluoroolefin according to claim 1, wherein the hydrofluorocarbon is at least one selected from the group consisting of 1,1,2,2-tetrafluoroethane and 1,1,1,2-tetrafluoroethane, and the compound A is trichloroethylene.

7. The method of producing a hydrofluoroolefin according to claim 1, wherein a molar ratio of the compound A to the hydrofluorocarbon contained in the mixture is from 0.05 to 0.99.

8. The method of producing a hydrofluoroolefin according to claim 1, wherein the mixture and the catalyst are contacted with each other at a temperature from 100 to 800° C.

9. The method of producing a hydrofluoroolefin according to claim 1, wherein a contact time between the mixture and the catalyst is from 0.1 to 100.0 seconds.

10. The method of producing a hydrofluoroolefin according to claim 1, further comprising drying the catalyst before contacting the mixture with the catalyst.

11. The method of producing a hydrofluoroolefin according to claim 1, wherein a concentration of water in the mixture is less than 500 ppm.

12. The method of producing a hydrofluoroolefin according to claim 1, further comprising:
generating the hydrofluoroolefin and hydrogen fluoride by a dehydrofluorination reaction of the hydrofluorocarbon; and
causing the compound A to act on the hydrogen fluoride generated by the dehydrofluorination reaction of the hydrofluorocarbon.

13. The method of producing a hydrofluoroolefin according to claim 12, wherein, causing the compound A to act on the hydrogen fluoride generated by the dehydrofluorination reaction of the hydrofluorocarbon includes, in a case in which the compound A is a compound represented by the Formula (A1) or a compound represented by the Formula (A2), a hydrogen fluoride addition reaction occurring in which hydrogen fluoride is added to the compound A, and in a case in which the compound A is a compound represented by the Formula (A3), a halogen exchange reaction occurring in which a halogen atom other than a fluorine atom in the compound A is exchanged with a fluorine atom of the hydrogen fluoride.
